(19) United States
(12) Patent Application Publication
(10) Pub. No.: US 2025/0205500 A1
(43) Pub. Date: Jun. 26, 2025

(54) ENDOVASCULAR NEURAL INTERFACE FOR BRAIN AND SPINAL CORD APPLICATIONS

(71) Applicant: Cerebrolytics Inc, Chicago, IL (US)

(72) Inventors: Kris SIEMIONOW, Chicago, IL (US); Paul LEWICKI, Tulsa, OK (US)

(21) Appl. No.: 19/077,415

(22) Filed: Mar. 12, 2025

Related U.S. Application Data
(63) Continuation-in-part of application No. 17/467,299, filed on Sep. 6, 2021, now abandoned.
(60) Provisional application No. 63/075,192, filed on Sep. 6, 2020.

(51) Int. Cl. A61N 1/372; A61M 37/00; A61N 1/36; A61N 1/378

(52) U.S. Cl. CPC A61N 1/37205; A61M 37/0015; A61N 1/3606; A61N 1/36062; A61N 1/36139; A61N 1/37223; A61N 1/3787; A61M 2037/0023; A61M 2205/04

(57) ABSTRACT

A communicating device for placing within a blood vessel includes a proximal end part; a terminal end part; and a wired connector that communicatively connects the proximal end part with the terminal end part. The wired connector has a length sufficient to position the proximal end part beneath a skin surface and the terminal end part at a target location within a blood vessel associated with neural tissue, and the wired connector comprises sensors distributed along its length to sense blood vessel parameters in a region between the proximal end part and the terminal end part. The proximal end part includes a battery connected to a wireless charging terminal and a signal processing circuit connected to a wireless communication terminal. The terminal end part includes a plurality of sensors, electrodes, and microfilaments configured to be deployed through walls of the blood vessel.

### CROSS-REFERENCE TO RELATED APPLICATIONS

This application is a continuation-in-part of U.S. patent application Ser. No. 17/467,299 filed on 6 Sep. 2021, entitled “A BRAIN-COMPUTER INTERFACE,” which is incorporated herein by reference in its entirety.

### TECHNICAL FIELD

This disclosure is related to the field of medical devices, in particular to endovascular-based neural interfaces that facilitate sensing and stimulation of brain or spinal cord tissue via a device positioned within a blood vessel.

### BACKGROUND

Many people suffer from pharmacologically resistant depression, epilepsy, addiction, and other neurological conditions. Moreover, to date there is no cure for neurodegenerative disorders such as Alzheimer's or Parkinson's. State-of-the-art understanding of these brain disorders is often limited to observational and postmortem analysis, and currently, obtaining real-time data from a diseased brain is very limited. Similarly, there are significant challenges in diagnosing and treating disorders involving the spinal cord, including pain management, paralysis, and injuries affecting motor and sensory function, where real-time monitoring and targeted modulation of spinal cord activity could potentially improve clinical outcomes.

Neural interfaces have been widely explored for applications in neuroprosthetics, paralysis treatment, and neuromodulation. Traditional interfaces often require direct implantation of electrodes onto neural tissue, which carries risks of fibrosis, foreign body reactions, and limited longevity. By contrast, vascular-based neural interfaces offer a promising alternative, enabling access to neural structures via the bloodstream, thereby reducing surgical invasiveness and potentially improving long-term device viability. In the intracranial context, devices placed in blood vessels near or within the brain can provide stimulation or sensing functions, for instance, in patients suffering from epilepsy, depression, addiction, or neurodegenerative diseases. Similarly, endovascular approaches targeting spinal cord vasculature can be used to deploy microelectrodes or microneedles, enabling selective interaction with spinal neural tissue for pain management, motor function restoration, or other therapeutic purposes.

A PCT application WO2017070252 discloses devices, methods, and systems for transmitting signals through a device located in a blood vessel of an animal, for stimulating and/or sensing activity of media proximal to the device, wherein the media includes tissue and/or fluid. The device comprises a frame structure forming a plurality of struts, where the frame structure is movable between a reduced profile and an expanded profile in which a diameter of the frame structure increases. At least one of the plurality of struts forming the frame structure comprises an electrically conductive material on a support material, the electrically conductive material extending along at least a portion of the strut and being covered with a non-conductive material. At least one electrode is formed by an opening in the non-conductive material on the portion of the strut. A lead is located at an end of the frame structure and configured to be in electrical communication with the electrically conductive portion, the lead extending from the frame structure.

### SUMMARY

There is a need to further develop the area of brain-computer interface to overcome several technical disadvantages of state of the art.

In one aspect, the invention relates to a communicating device for placing within a blood vessel, the communicating device comprising: a proximal end part; a terminal end part; a wired connector that communicatively connects the proximal end part with the terminal end part, wherein the wired connector has a length sufficient to position the proximal end part beneath a skin surface and the terminal end part at a target location within a blood vessel associated with neural tissue, and wherein the wired connector comprises sensors distributed along its length to sense blood vessel parameters in a region between the proximal end part and the terminal end part; wherein the proximal end part comprises a battery connected to a wireless charging terminal and a signal processing circuit connected to a wireless communication terminal; and wherein the terminal end part comprises a plurality of sensors, electrodes, and microfilaments configured to be deployed through walls of the blood vessel.

The terminal end part can be configured to be placed within an intracranial blood vessel system.

The terminal end part can be configured to be placed within a spinal cord vasculature system.

The proximal end part may have a diameter greater than the diameter of the terminal end part.

The proximal end part can be configured to be placed in a jugular vein.

The proximal end part can be configured to be placed in a vein in an arm or a leg.

The proximal end part can be connected to an antenna.

The microfilaments may have a diameter of about 5 to about 50 micrometers and a length of about 100 to about 500 micrometers, thereby permitting penetration of the vessel wall without significant vascular trauma.

The terminal end part may comprise a collapsible and expandable stent or a partial frame configured to anchor the microfilaments in place along the vessel wall.

The microfilaments can be microneedles configured to detect neural signals, deliver stimulation, or administer neuromodulatory substances to adjacent neural tissue.

The device may further comprise an introducer device configured to navigate the terminal end part through a femoral or jugular vein to the target location within the neural vasculature.

The terminal end part may further comprise a micro-robotic deployment mechanism arranged to insert the microfilaments through the wall of the blood vessel and into surrounding neural tissue.

The wired connector may comprise additional sensors to monitor chemical or electrical parameters of the blood vessel between the proximal end part and the terminal end part.

The terminal end part may comprise a collapsible and expandable stent for affixing the terminal end part at a desired location and a sleeve that houses the sensors, the electrodes and the microfilaments.

The proximal end part may comprise a stent that anchors the proximal end part within the blood vessel, and a sleeve that houses the battery, the wireless charging terminal, the signal processing circuit, and the wireless communication terminal.

The terminal end part may comprise a micro-robotic device configured to deploy microfilaments through the walls of the vessel and into the surrounding neural structures. The microfilaments may have a form of microneedles.

The microfilaments can be housed in an expandable and collapsible balloon inserted into the stent.

A single proximal end part can be communicatively coupled with a plurality of terminal end parts located at different positions within intracranial and/or spinal cord vasculature.

The microfilaments can be spring-loaded and protected by a resorbable sheath, such that removal or dissolution of the sheath causes the microfilaments to deploy and engage the neural tissue.

The microfilaments may have a form of barbs on hooks outside the stent.

At least some of the microfilaments may comprise barbed or hooked tips configured to facilitate tissue engagement and stable positioning within the neural tissue.

Surfaces of the microfilaments or electrodes can be functionalized with bioactive or biocompatible coatings to reduce immune response and improve signal fidelity.

The device may further comprise a fluid reservoir positioned in or near the terminal end part, wherein at least one of the microfilaments is configured to deliver a therapeutic fluid through the vessel wall to adjacent neural tissue.

The proximal end part may further comprise at least one on-board sensor configured to monitor vascular integrity or blood flow for real-time detection of potential complications from microfilament deployment.

The signal processing circuit can be configured to provide closed-loop neuromodulation by analyzing signals received from the terminal end part and automatically adjusting stimulation parameters in real time.

In another aspect, the invention relates to a method of using a communicating device for placing within a blood vessel, the communicating device comprising a proximal end part, a terminal end part, and a wired connector that communicatively connects the proximal end part with the terminal end part, wherein the terminal end part comprises a plurality of sensors, electrodes, and microfilaments, the method comprising: introducing the terminal end part into a blood vessel associated with neural tissue via an introducer device; positioning the terminal end part at a target location; and deploying the microfilaments so that they penetrate through the vessel wall to sense or stimulate the adjacent neural tissue.

Introducing the terminal end part may comprise navigating through a femoral or jugular vein to position the device either within an intracranial blood vessel or a spinal cord vasculature system.

The method may further comprise collecting real-time electrical or chemical data from the sensors located on or near the microfilaments, processing the data via the signal processing circuit at the proximal end part, and transmitting the processed data to an external controller.

The device and method have various applications. For example, the terminal end can be positioned in superficial cortical veins to stimulate motor cortex (in case of functional deficits of a patient) or in internal jugular vein to stimulate anterior nucleus (in case of a patient suffering from epilepsy, Alzheimer's). Furthermore, the terminal end can be positioned in the anterior communicating artery to interact with nucleus acumbens, subgenual cingulate white matter (depression), ventral capsule (obsessive compulsive disorder, addiction, depression).

By leveraging endovascular access to the spinal cord, the present system addresses a range of neurological and orthopedic indications, including but not limited to: paralysis treatment (by linking spinal motor circuits to external controllers or exoskeletons), pain modulation (by providing precise neuromodulation for spinal cord injury-related pain or chronic pain syndromes), neurorehabilitation (by enhancing post-injury recovery through targeted electrical stimulation protocols) or brain-computer interface extension (by augmenting brain-controlled prosthetics or exoskeletons with direct spinal feedback, thereby enabling more natural motor and sensory integration).

These and other features, aspects and advantages of the invention will become better understood with reference to the following drawings, descriptions, and claims.

### BRIEF DESCRIPTION OF DRAWINGS

Various embodiments are herein described, by way of example only, with reference to the accompanying drawings, wherein:

FIG. 1 shows schematically an embodiment for a communicating device for placing within a blood vessel;

FIG. 2 shows the device of FIG. 1 placed within the blood vessel;

FIG. 3 shows a more detailed schematic view of a terminal end part of the device of FIG. 1;

FIG. 4 shows positioning of a proximal end part of the device of FIG. 1;

FIG. 5 shows a functional schematic of the device;

### DETAILED DESCRIPTION OF THE EMBODIMENTS

The following detailed description is of the best currently contemplated modes of carrying out the invention. The description is not to be taken in a limiting sense but is made merely for the purpose of illustrating the general principles of the invention.

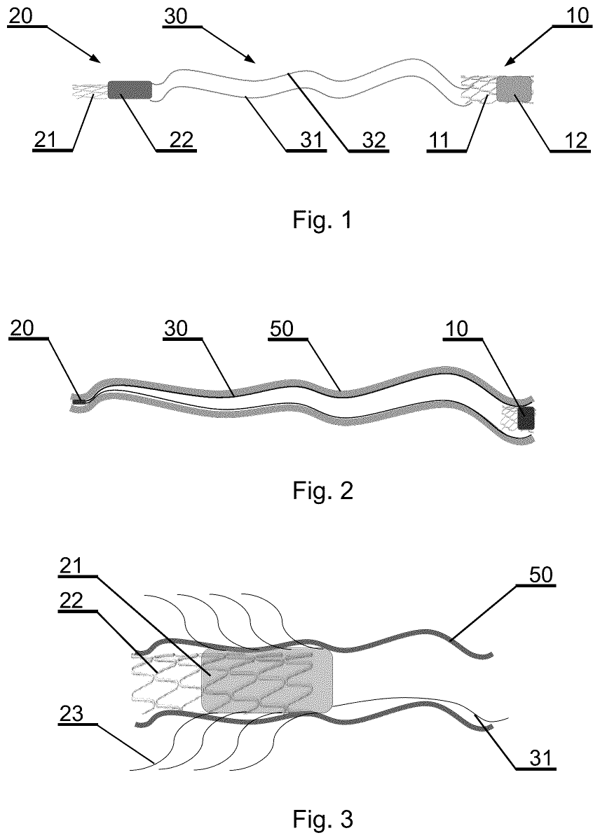

The communicating device for placing within a blood vessel has a structure as shown in an example in FIG. 1 and functional elements as shown in an example in FIG. 5. The device comprises a proximal end part 10, a terminal end part 20 and a wired connector 30 that connects the proximal end part 10 with the terminal end part 20.

In one embodiment, the device is supposed to be placed within a blood vessel, in particular within one of intracranial blood vessels, such as internal cerebral vein, great cerebral vein, basal veins, inferior anastomotic vein, superficial middle cerebral vein, superior anastomotic vein, thalamostriate veins, cortical vein, ophthalmic vein.

In another embodiment, an analogous design as explained with reference to the intracranial application, can be used for a spinal cord interface. In the spinal context, the device may be deployed in vessels associated with the spinal cord, such as segmental spinal cord vessels, including anterior spinal artery, posterior spinal veins, or radiculomedullary arteries. The purpose in this spinal context is to provide an endovascular interface enabling microelectrodes, located on microneedles, to penetrate the vessel wall and establish electrical communication with the spinal cord neural tissue.

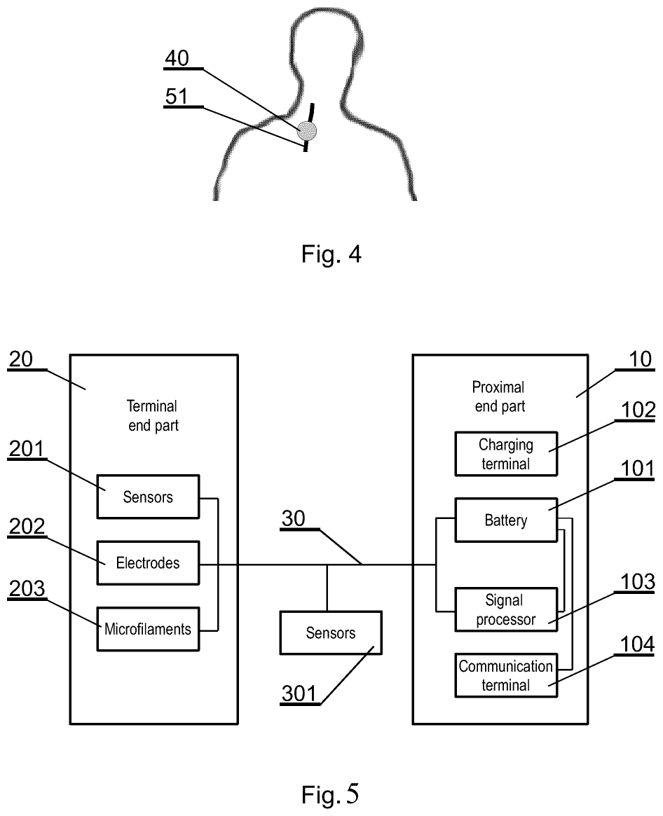

The device can be placed within the blood vessel by an introducer device, such as a steerable guidewire capable of traversing complex vasculature anatomy, that deploys the terminal end part 20 at a desired location within the intracranial blood vessels system and deploys the proximal end part 10 at a jugular vein 51 as shown in FIG. 4. In a spinal context, the same or similar approach is used, but navigation is performed to reach the target spinal cord vessel by a femoral or jugular vein access, guided by imaging such as fluoroscopy, intravascular ultrasound, or MRI. Other locations for the terminal end part 20 are possible as well-preferably, these are locations that are close to the skin surface to allow for communication and charging, such as veins in an arm or leg close to skin surface that are particularly preferable because these locations can be covered by smart clothes.

The terminal end part 20 is configured to sense and/or stimulate the anatomy parts at its vicinity, to provide stimulation, navigation (e.g., infrared and EM based) and/or guidance (e.g., AI based). It comprises a collapsible and expandable stent 21 and a sleeve 22. The stent 21 has a form of a net that can adjust its diameter so that it becomes adjacent to the internal walls of the blood vessel wherein the terminal end part 20 is positioned, so that it functions as an anchor for affixing the terminal end part 20 at a desired location. The sleeve 22 has a cylindrical shape, for example of a diameter of about 3 mm and a length of about 10 mm. The sleeve 22 houses various sensors 201 and electrodes 202 to sense and/or stimulate the target area. In particular, the sleeve 22 houses a micro-robotic device (i.e., an endovascular robot) that, when coupled to the introducer, deploys microfilaments 23, 203 through the walls of the vessel 50 and into the surrounding neural structures. For example, this can be done by expanding a balloon inserted into the center of the stent that deploys microneedles, and next collapsing and removing the balloon. Alternatively, the microneedles can be spring loaded and protected by a sheath, and when the sheath is removed or dissolved with time (e.g., the sheath can be made from polyglycolic acid or some other biocompatible material), the needles spring out from the stent, pierce the vessel and enter the brain matter. In a spinal application, the microneedles are designed to penetrate the vessel wall along a defined segment of the spinal cord vasculature, with lengths ranging, for example, from about 100 to 500 micrometers, and diameters of about 5 to 50 micrometers, in order to minimize vascular trauma while reaching the target spinal cord tissue. Furthermore, the needles can have a form of barbs on hooks outside the stent, while the stent can be advanced past its ideal position and then pulled back which makes the microneedles catch the tissue and then get pushed into the vessel wall, and through it, as the stent is pulled back. The microfilaments may have a form of microneedles, such as described in a publication “Microneedles: A smart approach and increasing potential for transdermal drug delivery system” by Waghule, Tejashree, et al. in Biomedicine & pharmacotherapy 109 (2019): 1249-1258. The microfilaments interact by picking up and delivering electrical signals. Therefore, some of the microfilaments can be electroconductive. Therefore, some of the microfilaments can be used to detect the brain waves or brain activity, while some of the microfilaments can be used to deliver neurotransmitters like dopamine or serotonin. Likewise, in the spinal application, the microfilaments can be used to record or stimulate activity in the spinal cord and deliver neuromodulatory substances if needed. The microfilaments 23, 203 allow to interface with a large cerebral surface area or with extended regions of the spinal cord, depending on the deployment location.

The proximal end part 10 comprises a collapsible and expandable stent 11 and a sleeve 12. The stent 11 has a form of a net that can adjust its diameter so that it becomes adjacent to the internal walls of the blood vessel wherein the proximal end part 10 is positioned, so that it functions as an anchor for affixing the proximal end part 10 at a desired location. The sleeve 12 has a cylindrical shape, for example of a diameter of about 10 mm and a length of about 20 mm. The sleeve 12 houses a battery 101, a charging terminal 102, signal processing circuits 103 and signal transmitting terminal 104. The signal processing circuit 103 collects data from the sensors 201 and process them e.g., to form data packets for transmission. Further, the signal processing circuit 103 reads incoming data to correspondingly control the electrodes 202. The charging terminal 102 and the transmitting terminal 104 may be of a wireless type and they can be connected to an antenna 40 that is located in a vicinity of the proximal end part 10. The antenna 40 allows communication with an external charging and signal transmission circuitry. Positioning of the proximal end part 10 in the jugular vein makes the proximal end part 10 and the antenna 40 easily accessible, as it is close to the skin surface. Alternatively, for a spinal cord application, the device can be navigated through the femoral vein until an intravascular location is reached where charging and communication remain feasible. The battery can be wirelessly recharged by inductive or ultrasound-based mechanisms, ensuring that the device has a stable power supply for both sensing and stimulation in neural or spinal contexts. A single proximal end part 10 may be communicatively coupled with a plurality of terminal end parts 20, each terminal end part 20 located at a different location within the brain or the spinal cord vasculature.

The wired connector 30 has a form of wires 31 that transmit power and signals between the proximal end part 10 and the terminal end part 20. The wired connector 30 may further comprise various sensors 301 so as to sense the vessel in a region between the locations of the end parts 10, 20. The sensors 201, 301 used in the terminal end part 20 and/or the wired connector 30 may be of various types. Non-limiting examples of sensors include sensors for measurement of dopamine, N-acetylaspartate (NAA), creatine, glutamate, choline, lactate, glutamate, myo-inositol, homovanilic acid, glucose, cholesterol, aminoacids: alanine, valine, leucine and isoleucine, ghrelin, leptin, electrolytes: Na+, K+, Ca++, Mg++. In spinal applications, these same or similar sensors may be positioned along the spinal cord segment to collect local chemical and electrical data that inform neuromodulation strategies.

In certain embodiments targeting spinal cord vasculature, the device includes a microstent or partial-frame structure specifically dimensioned and composed to match the smaller vessels associated with the spinal cord. The microstent or partial frame may have a diameter ranging from about 0.5 mm to about 2.5 mm and a length ranging from about 5 mm to about 30 mm, ensuring adequate coverage of a relevant spinal segment. Suitable materials for this stent/frame include nitinol, shape-memory alloys, or biocompatible polymer-coated metallic frameworks, which provide both flexibility and structural integrity. When deployed, the microstent or partial frame self-expands (or is balloon-expanded) to anchor itself within the vessel wall, facilitating stable microneedle placement.

The implantable power source (e.g., battery) housed in the proximal end part may exhibit a capacity from about 10 mAh to about 100 mAh, optimized to support low-power neuromodulation and sensor readouts. With periodic recharging—accomplished wirelessly through inductive or ultrasound-based power transmission—the battery can maintain an operational lifespan of approximately 5-10 years. This longevity supports chronic implantation scenarios where frequent surgical interventions would be undesirable.

The microneedles or microfilaments are designed to penetrate the vessel wall only to the depth necessary to contact the target neural tissue without significant vascular trauma. Real-time feedback from onboard sensors can monitor blood flow, vessel wall integrity, and tissue impedance, thereby allowing the system to stop or reverse microneedle deployment if unsafe conditions (e.g., potential perforation or hemorrhage risk) are detected. Additional control may be provided via mechanical stops or software-based gating that limits needle extension beyond a preset distance.

To reduce immune response and enhance signal fidelity, the microfilaments and electrodes may be coated or functionalized with specialized materials. Non-limiting examples include platinum-iridium alloys, carbon nanotube composites, and silicon carbide films, which provide a stable, high-conductivity interface. Surface modifications such as polymer coatings can also limit fibrosis and preserve a high-quality neural interface over extended implantation periods.

In some implementations, the wireless communication module includes encryption protocols to secure bidirectional data transfer between the implanted device and an external controller. This safeguards patient data and ensures that commands for neuromodulation (e.g., stimulation parameters) cannot be intercepted or altered by unauthorized entities. Such secure, closed-loop neuromodulation can be critical when applying the device to conditions like epilepsy, spinal cord injuries, or neurodegenerative diseases.

Particulars of the components of the device that have not been elaborated in detail in this description are known to skilled persons and do not require to be further explained. A skilled person will realize e.g., what type of stent and sensors to use for the terminal end part, how to connect by wire the terminal end part with the proximal end part or how to wirelessly charge the battery and transmit data. By way of analogy, the same structural design of microstent, partial frames, and microneedle arrangement can be utilized for both cerebral and spinal use cases, with the adjustments of dimension and location dictated by the relevant vascular anatomy and target neural tissue.

In an exemplary method of using the device, an introducer or guidewire is first navigated through a blood vessel, such as a femoral vein or jugular vein, until it reaches the targeted region of the neural vasculature. Under appropriate imaging guidance (e.g., fluoroscopy, intravascular ultrasound, or magnetic resonance imaging), the user advances the terminal end part of the device until it is positioned in proximity to the desired neural tissue, whether in the intracranial venous system or in spinal cord vasculature. Once the terminal end part is in place, the collapsible stent or partial frame is expanded so that it makes contact with the vessel wall. The microfilaments, which can be microneedles, are then deployed through the vessel wall to interface with adjacent neural structures, picking up or delivering electrical signals as well as enabling the delivery of neuromodulatory substances if needed.

After deployment, the proximal end part remains positioned in a suitable location closer to the skin surface—commonly in a jugular vein, subclavian vein, or other accessible vessel—where it can more easily communicate through an antenna with external control systems. Power is supplied wirelessly via inductive or ultrasound-based charging mechanisms, and the signal processing circuit located in the proximal end part continuously collects, processes, and transmits data from the terminal end part to an external controller. In real time, the system can apply electrical stimulation to modulate activity in the targeted neural tissue, for example, in the treatment of neurological conditions such as epilepsy, depression, pain, spinal cord injury, or other disorders. This closed-loop neuromodulation allows the system to adjust stimulation parameters automatically based on feedback from the sensors, optimizing the therapeutic effect while minimizing adverse reactions.

During and after the procedure, various on-board sensors in both the wired connector and the proximal end part monitor parameters such as blood flow, tissue impedance, and local chemical concentrations. If the sensors detect potential complications, such as abnormal changes in vascular integrity or signal artifacts indicating microfilament displacement, the system can send an alert to the external controller. Clinicians may then adjust device settings, stimulation protocols, or, if necessary, reposition or retrieve the device under controlled conditions. This method of endovascular deployment, stable anchoring, and real-time data communication provides a minimally invasive means to achieve reliable, long-term sensing and stimulation within the brain or spinal cord.

If the method further involves delivering a therapeutic fluid, the microfilaments can be configured as hollow microneedles connected to a fluid reservoir located in or near the terminal end part. Under software or physician control, the device can release measured doses of neuromodulatory substances directly into the neural tissue. Integration of fluid delivery capability with electrical signal detection and stimulation enables a synergistic therapeutic approach. This combination of electrical, chemical, and mechanical interaction with neural circuits opens new possibilities for individualized treatment strategies for a wide range of neurological and spinal disorders.

While the invention has been described with respect to a limited number of embodiments, it will be appreciated that many variations, modifications, and other applications of the invention may be made. Therefore, the claimed invention as recited in the claims that follow is not limited to the embodiments described herein.

What is claimed is:

1. A communicating device for placing within a blood vessel, the communicating device comprising:
a proximal end part;
a terminal end part;
a wired connector that communicatively connects the proximal end part with the terminal end part, wherein the wired connector has a length sufficient to position the proximal end part beneath a skin surface and the terminal end part at a target location within a blood vessel associated with neural tissue, and wherein the wired connector comprises sensors distributed along its length to sense blood vessel parameters in a region between the proximal end part and the terminal end part;
wherein the proximal end part comprises a battery connected to a wireless charging terminal and a signal processing circuit connected to a wireless communication terminal; and
wherein the terminal end part comprises a plurality of sensors, electrodes, and microfilaments configured to be deployed through walls of the blood vessel.

2. The communicating device of claim 1, wherein the terminal end part is configured to be placed within an intracranial blood vessel system.

3. The communicating device of claim 1, wherein the terminal end part is configured to be placed within a spinal cord vasculature system.

4. The communicating device of claim 3, wherein the microfilaments have a diameter of about 5 to about 50 micrometers and a length of about 100 to about 500 micrometers, thereby permitting penetration of the vessel wall without significant vascular trauma.

5. The communicating device of claim 1, wherein the terminal end part comprises a collapsible and expandable stent or a partial frame configured to anchor the microfilaments in place along the vessel wall.

6. The communicating device of claim 1, wherein the microfilaments are microneedles configured to detect neural signals, deliver stimulation, or administer neuromodulatory substances to adjacent neural tissue.

7. The communicating device of claim 1, further comprising an introducer device configured to navigate the terminal end part through a femoral or jugular vein to the target location within the neural vasculature.

8. The communicating device of claim 1, wherein the terminal end part further comprises a micro-robotic deployment mechanism arranged to insert the microfilaments through the wall of the blood vessel and into surrounding neural tissue.

9. The communicating device of claim 1, wherein the wired connector comprises additional sensors to monitor chemical or electrical parameters of the blood vessel between the proximal end part and the terminal end part.

10. The communicating device of claim 1, wherein the proximal end part includes a stent that anchors the proximal end part within the blood vessel, and a sleeve that houses the battery, the wireless charging terminal, the signal processing circuit, and the wireless communication terminal.

11. The communicating device of claim 1, wherein a single proximal end part is communicatively coupled with a plurality of terminal end parts located at different positions within intracranial and/or spinal cord vasculature.

12. The communicating device of claim 1, wherein the microfilaments are spring-loaded and protected by a resorbable sheath, such that removal or dissolution of the sheath causes the microfilaments to deploy and engage the neural tissue.

13. The communicating device of claim 1, wherein at least some of the microfilaments comprise barbed or hooked tips configured to facilitate tissue engagement and stable positioning within the neural tissue.

14. The communicating device of claim 1, wherein surfaces of the microfilaments or electrodes are functionalized with bioactive or biocompatible coatings to reduce immune response and improve signal fidelity.

15. The communicating device of claim 1, further comprising a fluid reservoir positioned in or near the terminal end part, wherein at least one of the microfilaments is configured to deliver a therapeutic fluid through the vessel wall to adjacent neural tissue.

16. The communicating device of claim 1, wherein the proximal end part further comprises at least one on-board sensor configured to monitor vascular integrity or blood flow for real-time detection of potential complications from microfilament deployment.

17. The communicating device of claim 1, wherein the signal processing circuit is configured to provide closed-loop neuromodulation by analyzing signals received from the terminal end part and automatically adjusting stimulation parameters in real time.

18. A method of using a communicating device for placing within a blood vessel, the communicating device comprising a proximal end part, a terminal end part, and a wired connector that communicatively connects the proximal end part with the terminal end part, wherein the terminal end part comprises a plurality of sensors, electrodes, and microfilaments, the method comprising:
introducing the terminal end part into a blood vessel associated with neural tissue via an introducer device;
positioning the terminal end part at a target location; and
deploying the microfilaments so that they penetrate through the vessel wall to sense or stimulate the adjacent neural tissue.

19. The method of claim 18, wherein introducing the terminal end part comprises navigating through a femoral or jugular vein to position the device either within an intracranial blood vessel or a spinal cord vasculature system.

20. The method of claim 18, further comprising collecting real-time electrical or chemical data from the sensors located on or near the microfilaments, processing the data via the signal processing circuit at the proximal end part, and transmitting the processed data to an external controller.
